(19) United States
(12) Patent Application Publication
(10) Pub. No.: US 2024/0315975 A1
(43) Pub. Date: Sep. 26, 2024

(54) SOLID SELF-EMULSIFYING PHARMACEUTICAL COMPOSITIONS

(71) Applicant: AUSCANN GROUP HOLDINGS LTD, Joondalup (AU)

(72) Inventors: Paul D.R MACLEMAN, Loch (AU); Musabek MAVLIANOV, Blackburn North (AU)

(73) Assignee: AUSCANN GROUP HOLDINGS LTD, Joondalup (AU)

(21) Appl. No.: 18/592,160

(22) Filed: Feb. 29, 2024

Related U.S. Application Data
(63) Continuation of application No. 17/263,645, filed on Jan. 27, 2021, now abandoned.

(30) Foreign Application Priority Data
Jul. 31, 2018 (AU) 2018902782

(51) Int. Cl. A61K 9/48; A61K 9/00; A61K 31/05; A61K 31/164; A61K 31/352

(52) U.S. Cl. CPC A61K 9/4808; A61K 9/0053; A61K 9/4816; A61K 9/485; A61K 9/4858; A61K 9/4866; A61K 9/4875; A61K 31/05; A61K 31/164; A61K 31/352

(57) ABSTRACT

The disclosure describes a solid self-emulsifying composition for oral solid dosages containing at least one cannabinoid and a method of preparing said solid self-emulsifying composition. The compositions as disclosed herein can be readily formulated into pharmaceutically acceptable oral solid dosage formulations suitable for administration to a subject in need of treatment, thereby providing certainty with regard to administration of a pre-defined therapeutically effective amount of the at least one cannabinoid.

### TECHNICAL FIELD

The disclosure relates to solid self-emulsifying pharmaceutical compositions comprising at least one cannabinoid and a method of preparing said compositions.

### BACKGROUND

The discussion of the background to the disclosure is intended to facilitate an understanding of the disclosure. However, it should be appreciated that the discussion is not an acknowledgement or admission that any of the material referred to was published, known or part of the common general knowledge as at the priority date of the application.

There is growing interest in the use of cannabis and cannabinoids for treatment of a wide range of medical conditions and disorders.

Currently, medicinal cannabis may be administered in several forms including capsules containing dried powdered cannabis plant material; edible ‘food’ products produced by infusing cannabis extract into a lipid phase (e.g. butter, cooking oil, edible fat) or a solvent phase (e.g. glycerol, glucose, alcohol); hard or soft-shell gelatin capsules containing cannabinoids dissolved in medium chain triglycerides or carrier oils; oil-based liquids containing cannabis extracts in various ratios and concentrations of cannabidiol (CBD) and delta-9-tetrahydrocannabinol (THC); chewing gum, inhalers produced by vaporisation of dried plant material; liquid sprays or aerosols by delivery to the oral mucosa; and as nutraceuticals combined with vitamins, minerals and other nutrients within lipid nanospheres.

The drawbacks of many of the above formulations include wide variability in the composition of active ingredients, poor stability and low bioavailability. Generally, these drawbacks arise because of the physicochemical properties of cannabinoids which are lipophilic, water-insoluble and typically exist as tacky, resinous tar-like substances comprising a complex mixture of diverse chemical compounds.

An effective oral delivery vehicle for cannabis and cannabinoids, in particular THC and CBD, providing consistent and stable dosages of the active ingredients with predictable bioavailability would be desirable.

Various embodiments as disclosed herein seek to overcome at least some of the aforementioned disadvantages.

### SUMMARY

The disclosure provides a solid self-emulsifying composition for oral solid dosages containing at least one cannabinoid, a method of preparing said solid self-emulsifying composition, and oral solid dosage formulations including said solid self-emulsifying composition.

Various embodiments of the disclosure provide a solid self-emulsifying composition for oral solid dosages comprising at least one cannabinoid, a lipophilic solvent, an emulsifier, and an adsorbent. It will be appreciated that the lipophilic solvent, the emulsifier and the adsorbent may be pharmaceutically-acceptable substances.

In one embodiment, at least one cannabinoid may be selected from a group comprising anandamide, 2-arachidonoylglycerol, cannabichromene (CBC), cannabichromenic acid (CBCA), cannabichormevarin (CBCV), cannabichromevarinic acid (CBCVA), cannabidiol (CBD), cannabidiolic acid (CBDA), cannabidivarin (CBDV), cannabidivarinic acid (CBDVA), cannabielsoin (CBE), cannabicyclol (CBL), cannabinodiol (CBND), cannabigerol (CBG), cannabigerolic acid (CBGA), cannabigerovarin (CBGV), cannabigerovarinic acid (CBGVA), cannabinol (CBN), cannabinolic acid (CBNA), cannabitriol (CBT), delta-8-tetrahydrocanninol, delta-8-tetrahydrocannabinolic acid, delta-9-tetrahydrocannabinol (THC), delta-9-tetrahydrocannabinolic acid (THCA), delta-9-tetrahydrocannabivarin (THCV), delta-9-tetrahydrocannabivarinic acid (THCVA), 11-nor-9-carboxy-delta-9-tetrahydrocannabinol (THCCOOCH), 11-nor-9-carboxy-delta-8-tetrahydrocannabinol, 11-hydroxy-delta-8-tetrahydrocannabinol, and 11-hydroxy-delta-9-tetrahydrocannabinol, dimethyl heptylpentyl cannabidiol (DMHP-CBD), 6,12-dihydro-6-hydroxy-cannabidiol, (3S,4R)-7-hydroxy-.DELTA.6-tetrahydrocannabinol homologs and derivatives, (+)-4-[4-DMH-2,6-diacetoxy-phenyl]-2-carboxy-6,6-dimethylbicyclo[3.1.1]he-pt-2-en, and other 4-phenylpinene derivatives, and cannabidiol (−)(CBD) analogs such as (−)CBD-monomethylether, (−)CBD dimethyl ether; (−)CBD diacetate; (−)3′-acetyl-CBD monoacetate, cannabinol propyl variant (CBNV), and nabilone.

In certain embodiments, the cannabinoid comprises cannabidiol (CBD).

In certain embodiments, the cannabinoid comprises delta-9-tetrahydrocannabinol (THC).

In certain embodiments, the cannabinoid comprises CBD and THC in a ratio of from 1:100 to 100:1, 1:10 to 10:1, 1:3 to 3:1, 1:2 to 2:1 or 1:1.

In certain embodiments, the lipophilic solvent comprises a vegetable oil, medium chain triglycerides, or a mixture thereof. Suitable examples of vegetable oils include, but are not limited to, cotton seed oil, safflower oil, sunflower oil, peanut oil, linseed oil, corn oil, olive oil, coconut oil, soybean oil, sesame oil, chia (Salvia Hispanica L.) seed oil, wheat germ oil, canola oil, castor oil, hydrogenated castor oil and any mixtures thereof. Examples of medium chain triglycerides that may be suitable for use in embodiments of the present disclosure comprise tricaproin, tricaprylin, tricaprin, trilaurin, and mixtures thereof. I

In certain embodiments, the emulsifier comprises a surfactant, in particular a non-ionic surfactant, in particular a polyethoxylated non-ionic surfactant. Suitable examples of polyethoxylated non-ionic surfactants include, but are not limited to, ethoxylated linear alcohols, ethoxylated alkyl-phenols, acid ethoxylated fatty acids, glycerol esters, esters of hexitols and cyclic anhydrohexitols. In one particular embodiment, the emulsifier comprises polyoxyl castor oil.

In one embodiment the adsorbent may comprise an inert particulate material. The inert particulate material may have a particle size in a range of from about 3 to 350 micron, in particular from about 20 micron to about 60 micron. The inert particulate material may be mesoporous with a mesopore volume in a range of from about 1.5 to about 1.9 mL/g. The inert particulate material may have a surface area of from about 150 to 350 m²/g, in particular from about 260 to about 320 m²/g.

In certain embodiments the inert particulate material may comprise amorphous silica.

In an alternative embodiment, the inert particulate material may comprise a pharmaceutically acceptable metal oxide. Suitable examples of pharmaceutically acceptable metal oxides include, but are not limited to, zinc oxide, titanium dioxide, cerium oxide and iron oxide.

In one embodiment, the solid self-emulsifying composition may further comprise an antioxidant, in particular a lipophilic antioxidant such as dl-α-tocopherol.

Various embodiments of the disclosure provide a method of preparing a solid self-emulsifying composition comprising:
a) providing a solution of at least one cannabinoid and an emulsifier in a liphophilic solvent;
b) mixing said solution with an adsorbent to produce the solid self-emulsifying composition.

In certain embodiments, the mixing step comprises adding said solution dropwise to the adsorbent with continuous stirring and blending the resulting mixture for a period sufficient to obtain the solid self-emulsifying composition. Advantageously, the solid self-emulsifying composition may be a free flowing powder. The rate of adding said solution to the adsorbent may be between 60-600 drops per minute. The rate of continuous stirring may be from 50 to 400 rpm. The mixture may be blended at a rate of 100-1000 rpm for 5 to 60 minutes. The mixture may be held between 30 min to 12 hours before further processing in order for droplets of the solution to settle within pores of the adsorbent.

In alternative embodiments, the mixing step comprises spraying the solution onto the adsorbent with continuous stirring and blending the resulting mixture for a period sufficient to obtain the solid self-emulsifying composition. The solid self-emulsifying composition may comprise a free-flowing powder or a crystalline powder.

In one embodiment, said solution comprises the least one cannabinoid and lipophilic solvent at a dilution factor from about 1:1 to about 1:90.

In certain embodiments, said solution comprises from about 3 wt % to about 40 wt % emulsifier.

In one embodiment, said solution may further comprise an antioxidant, in particular a lipophilic antioxidant such as dl-α-tocopherol. Said solution may comprise 0.02-1% dl-α-tocopherol.

In some embodiments, the method further comprises blending the solid self-emulsifying composition with one or more excipients.

Various embodiments of the disclosure also provide an oral dosage formulation comprising the solid self-emulsifying composition as defined above. The oral dosage formulation may be in an acceptable pharmaceutical form for oral administration. Suitable examples of such acceptable pharmaceutical forms include, but are not limited to a hard gelatine capsule, a soft gelatine capsule, hydroxypropyl methylcellulose (HPMC) capsules, pullulan capsules, a tablet, an effervescent tablet, a strip, a caplet, a sachet, a lozenge, a suspension, a suppository, a sub-lingual or buccal delivered form for local absorption, an effervescent powder, or a powder for a suspension.

In one embodiment, the oral dosage formulation may further comprise at least one pharmaceutical excipient selected from a group comprising fillers, binders, anti-caking agents, disintegrants, lubricants, glidants, antioxidants, colouring agents, coating agents, sweetening agents, sustained release agents.

In some embodiments, the CBD may be present in the oral dosage formulation in an amount from about 0.5 mg to 200 mg, or from about 2.5 mg to 20 mg.

In some embodiments, the THC may be present in the oral dosage formulation in an amount from about 0.5 mg to 200 mg, or from about 2.5 mg to 20 mg.

In some embodiments, the lipophilic solvent may be present in the oral dosage formulation in an amount from about 2 wt % to about 35 wt %.

In some embodiments, the emulsifier may be present in the oral dosage formulation in an amount of from about 1 wt % to 30 wt %.

In some embodiments, the adsorbent may be present in the oral dosage formulation in an amount from about 4 wt % to about 35 wt %.

In some embodiments, the antioxidant may be present in the oral dosage formulation in an amount from about 0.02 wt % to about 1.0 wt %.

In some embodiments, the filler and/or binder may be present in the oral dosage formulation in an amount from about 10 wt % to about 60 wt %.

In some embodiments, the anti-caking agent may be present in the oral dosage formulation in an amount from about 5 wt % to about 45 wt %.

Some embodiments of the disclosure relate to the solid self-emulsifying composition or the oral dosage formulation as defined above being used in the manufacture of a medicament for preventing, treating, and/or lessening the severity of a disease in a subject, wherein the disease is at least one of an inflammatory disorder, a neurological disorder, a psychiatric disorder, a malignancy, an immune disorder, a metabolic disorder, an infectious disease, a gastrointestinal disorder, a cardiovascular disorder, cancer, pain.

Some embodiments relate to a method of preventing, treating and/or lessening the severity of a disease in a subject, wherein the disease is at least one of an inflammatory disorder, a neurological disorder, a psychiatric disorder, a malignancy, an immune disorder, a metabolic disorder, an infectious disease, a gastrointestinal disorder, a cardiovascular disorder, cancer, pain said method comprising administering to a subject in need thereof an effective amount of the solid self-emulsifying composition or the oral dosage formulation as defined above.

Some embodiments relate to a kit comprising at least one container comprising a pre-defined amount of the solid self-emulsifying composition as defined above.

Some embodiments relate to a method of providing a plasma concentration of at least one cannabinoid in a pre-determined concentration range in a subject comprising administering to the subject a pre-determined amount of the solid self-emulsifying composition or the oral dosage formulation as defined above.

Some embodiments relate to a method for improving the pharmacokinetic profile of at least one cannabinoid comprising administering to the subject in need of such treatment an effective amount of the solid self-emulsifying composition or the oral dosage formulation as defined above.

### DESCRIPTION OF EMBODIMENTS

The disclosure relates to solid self-emulsifying composition comprising at least one cannabinoid, oral solid dosage formulations of said solid self-emulsifying composition and a method of preparing said solid self-emulsifying composition.

### General Terms

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The term “about” as used herein means within 5%, and more preferably within 1%, of a given value or range. For example, “about 3.7%” means from 3.5 to 3.9%, preferably from 3.66 to 3.74%. When the term “about” is associated with a range of values, e.g., “about X % to Y %”, the term “about” is intended to modify both the lower (X) and upper (Y) values of the recited range. For example, “about 20% to 40%” is equivalent to “about 20% to about 40%”.

All percentages by weight in the compositions are percentages by weight with respect to the whole composition.

### Specific Terms

The term “self-emulsifying composition” as used herein refers to an isotropic mixture of a liquid or semisolid active ingredient, oil/lipophilic solvent, surfactant and/or co-surfactant which form fine emulsion/liquid droplets, on dilution with a physiological fluid.

The term “pharmaceutically acceptable” with respect to a substance as used herein means that substance which is suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “therapeutically effective amount” as used herein refers to an amount of active ingredient needed to provide a desired level of active ingredient in the bloodstream or at a target organ of to provide an anticipated physiological response. The precise amount will vary in response to several factors including, but not limited to, the type of active ingredient, bioavailability of the active ingredient, patient characteristics (e.g. age, weight, gender), severity of symptoms, contraindications, and so forth. A therapeutically effective amount of an active ingredient may be administered in a single dosage, or through multiple dosages of an amount that cumulatively provides a therapeutic effect. The ‘therapeutic effect’ may reduce the severity of a disease, medical condition or one or more associated symptoms, and/or may be therapeutic in terms of a partial or complete cure of a disease or medical condition.

### Solid Self-Emulsifying Compositions

Various embodiments of the disclosure provide a solid self-emulsifying composition for oral solid dosage formulations comprising at least one cannabinoid, a lipophilic solvent, an emulsifier, and an adsorbent.

The solid self-emulsifying compositions as disclosed herein provide the at least one cannabinoid in a solid form as a free flowing powder that can be stably maintained. The ability to provide the at least one cannabinoid in solid form as a free flowing powder is advantageous for its pharmaceutical use because cannabinoids have lipophilic/hydrophobic properties which are problematic with respect to dissolution and bioavailability. The compositions as described herein have better dissolution properties and thus improved bioavailability upon administration to a subject in need of treatment.

The compositions as disclosed herein are also advantageous because they can be readily formulated into pharmaceutically acceptable oral solid dosage formulations suitable for administration to a subject in need of treatment, thereby providing certainty with regard to administration of a pre-defined therapeutically effective amount of the at least one cannabinoid.

In contrast, currently available compositions containing one or more cannabinoids are available in liquid form, which is unstable and inconvenient to formulate in pre-defined dosages.

Cannabinoids are also currently available in a dried powdered form of cannabis plant material or as a resinous extract. In these particular forms, the active ingredient(s) of interest may be present in varying amounts and therefore it is difficult to administer a pre-defined therapeutically effective dosage. Additionally, a plurality of cannabinoids as well as other cannabis plant constituents such as terpenes, sesquiterpenes, carotenes, flavonoids are also present and are co-administered together with the active ingredient(s) of interest. Consequently, the anticipated therapeutic effect of the active ingredient(s) may be considerably altered.

The term “cannabinoid” as used herein refers to a class of C₂₁ terpenophenolic compounds that represent a group of compounds found in Cannabis sativa. The term encompasses synthetic analogues of such C₂₁ terpenophenolic compounds.

The at least one cannabinoid may be selected from a group comprising anandamide, 2-arachidonoylglycerol, cannabichromene (CBC), cannabichromenic acid (CBCA), cannabichormevarin (CBCV), cannabichromevarinic acid (CBCVA), cannabidiol (CBD), cannabidiolic acid (CBDA), cannabidivarin (CBDV), cannabidivarinic acid (CBDVA), cannabielsoin (CBE), cannabicyclol (CBL), cannabinodiol (CBND), cannabigerol (CBG), cannabigerolic acid (CBGA), cannabigerovarin (CBGV), cannabigerovarinic acid (CBGVA), cannabinol (CBN), cannabinolic acid (CBNA), cannabitriol (CBT), delta-8-tetrahydrocanninol, deleta-8-tetrahydrocannabinolic acid, delta-9-tetrahydrocannabinol (THC), delta-9-tetrahydrocannabinolic acid (THCA), delta-9-tetrahydrocannabivarin (THCV), delta-9-tetrahydrocannabivarinic acid (THCVA), 11-nor-9-carboxy-delta-9-tetrahydrocannabinol (THCCOOCH), 11-nor-9-carboxy-delta-8-tetrahydrocannabinol, 11-hydroxy-delta-8-tetrahydro-cannabinol, and 11-hydroxy-delta-9-tetrahydrocannabinol, dimethyl heptylpentyl cannabidiol (DMHP-CBD), 6,12-dihydro-6-hydroxy-cannabidiol, (3S,4R)-7-hydroxy-.DELTA.6-tetrahydrocannabinol homologs and derivatives, (+)-4-[4-DMH-2,6-diacetoxy-phenyl]-2-carboxy-6,6-dimethylbicyclo[3.1.1]he-pt-2-en, and other 4-phenylpinene derivatives, and cannabidiol (−)(CBD) analogs such as (−)CBD-monomethylether, (−)CBD dimethyl ether; (−)CBD diacetate; (−)3′-acetyl-CBD monoacetate, cannabinol propyl variant (CBNV), and nabilone.

The solid self-emulsifying composition may comprise cannabidiol (CBD), delta-9-tetrahydrocannabinol (THC), or a mixture of CBD and THC in a ratio which may vary from 1:100 to 100:1, 1:10 to 10:1, or 1:1.

Assayed amounts of the at least one cannabinoid are provided in the lipophilic solvent. In particular the at least one cannabinoid in the lipophilic solvent is present at a dilution factor of from about 1:1 to about 1:90. The lipophilic solvent comprises a vegetable oil, medium chain triglycerides, or a mixture thereof. Examples of vegetable oils that may be suitable for use in embodiments of the present disclosure comprise cotton seed oil, safflower oil, sunflower oil, peanut oil, linseed oil, corn oil, olive oil, coconut oil, soybean oil, sesame oil, chia (Salvia Hispanica L.) seed oil, wheat germ oil, canola oil, castor oil, hydrogenated castor oil and any mixtures thereof.

The term “medium-chain triglycerides (MCTs) as used herein refers to triglycerides whose fatty acids have an aliphatic tail of 6-12 carbon atoms. Examples of medium chain triglycerides that may be suitable for use in embodiments of the present disclosure comprise tricaproin, tricaprylin, tricaprin, trilaurin, and mixtures thereof. It will be appreciated that the term “medium-chain triglycerides” also encompasses a mixture of triglycerides of saturated fatty acids with 8 and 10 carbon atoms such as of caprylic (octanoic) acid and of capric (decanoic) acid.

Inclusion of the emulsifier in the composition facilitates formation of a liquid self-emulsifying composition. The liquid self-emulsifying composition is typically prepared by dissolving at least one cannabinoid in the lipophilic solvent and mixing or blending the solution with the emulsifier.

The emulsifier may be a surfactant, in particular a non-ionic surfactant. The term “non-ionic surfactant” as used herein refers to an organic compound having covalently bonded oxygen-containing hydrophilic groups which are bonded to hydrophobic parent structures, and which are capable of lowering the surface tension between two non-miscible liquids, in particular a hydrophilic liquid and a hydrophobic liquid.

In particular, the emulsifier may be a polyethoxylated non-ionic surfactant. Examples of polyethoxylated non-ionic surfactants suitable for use in the compositions disclosed herein comprise ethoxylated linear alcohols, ethoxylated alkyl-phenols, acid ethoxylated fatty acids, glycerol esters, esters of hexitols and cyclic anhydrohexitols. In particular the emulsifier may be polyoxyl castor oil.

The emulsifier also facilitates the dispersion of the lipophilic solution of the at least one cannabinoid at the molecular level in the adsorbent to provide the solid self-emulsifying composition as disclosed herein. The term “solid self-emulsifying composition” as used herein refers to a solid phase of a liquid self-emulsifying composition in the form of powders or nanoparticles suitable for use in oral solid dosage formulations. The compositions are characterised by their flowability characteristics which allow them to be formulated into oral solid dosage formulations.

The solid self-emulsifying composition may comprise from about 3 wt % to about 40 wt % emulsifier.

The adsorbent may comprise an inert particulate material. For example, the inert particulate material may comprise amorphous silica. The term “amorphous” as used herein refers to a non-crystalline state. Suitable examples of amorphous silica include, but are not limited to, colloidal amorphous silica sold under the trade name Aeroperl 300 Pharma grade, Syloid 244 FP Silica, Syloid XDP Silica, the Supernat range of silica, or the Aerosil range of Colloidal Silicon Dioxide.

Alternatively, the inert particulate material may be a pharmaceutically acceptable metal oxide. Suitable examples of pharmaceutically acceptable metal oxides include, but are not limited to, zinc oxide, titanium dioxide, cerium oxide and iron oxide.

In another embodiment, the inert particulate material may comprise microcrystalline cellulose, silicified microcrystalline cellulose, powdered cellulose, starch, dextrose, polysaccharides and dextrates.

The inert particulate material may have a particle size in a range of from about 20 micron to about 60 micron. The distribution of particle size may be measured using optical microscopy, laser diffraction particle size analysis, dynamic light scattering, imaging particle analysis or other techniques which are known to those of skill in the art.

The inert particulate material may be mesoporous with a mesopore volume in a range of from about 1.5 to about 1.9 mL/g. The term “mesoporous” as used herein refers to pores ranging in size from about 2 nm to about 100 nm. The pores are categorized as “open pores” that connect through and open onto a surface of a particle or as “closed pores” that are sealed from fluid ingress from the surface of the particle. The distribution of pore sizes and total pore volume of the particle may be measured using gas adsorption and pycnometry or other techniques which are known to those of skill in the art.

The inert particulate material may have a surface area of from about 260 to about 320 m²/g.

The weight ratio of adsorbent to liquid self-emulsifying composition may be from about 1:1.0 to about 1:2, in particular from about 1:1.5 to about 1:1.75, even from about 1:1.588 to about 1:1.65.

The solid self-emulsifying composition may further comprise an antioxidant to increase the stability, in particular a lipophilic antioxidant such as dl-α-tocopherol. The antioxidant may be present in said composition in an amount from about 0.02 wt % to about 1.0 wt %.

The solid self-emulsifying composition as described herein may take the form of a free flowing powder or a crystalline powder and oral dosage formulations may be conveniently formulated in an acceptable pharmaceutical form for oral administration. Suitable examples of such acceptable pharmaceutical forms include, but are not limited to a hard gelatine capsule, a soft gelatine capsule, hydroxypropyl methylcellulose (HPMC) capsule, pullulan capsule, a tablet, an effervescent tablet, a strip, a caplet, a sachet, a lozenge, a suspension, a suppository, a sub-lingual or buccal delivered form for local absorption, an effervescent powder, or a powder for a suspension.

Generally the oral dosage formulation may further comprise at least one pharmaceutical excipient selected from a group comprising fillers, binders, anti-caking agents, disintegrants, lubricants, glidants, preservatives, antioxidants, surfactants, effervescent excipients, colouring agents, coating agents, sweetening agents, sustained release agents and so forth for their customary purposes and in typical amounts without adversely affecting the properties of the compositions.

Suitable examples of fillers and binders include, but are not limited to, lactose, mannitol, xylitol, microcrystalline cellulose, methyl cellulose, dibasic calcium phosphate (anhydrous and dihydrate), starch, and any combinations thereof.

An anti-caking agent may be included to prevent the formation of lumps (caking) and to assist flowability properties of the oral solid dosage formulation. Suitable examples of anti-caking agents include, but are not limited to, silicon dioxide, lactose, tricalcium phosphate, and any combination thereof.

Disintegrants may be added to oral solid dosage formulations to aid in their deaggregation and to cause rapid break-up of the solids when they come into contact with moisture. Suitable examples of disintegrants include, but are not limited to, corn starch, potato starch, sodium starch glycolate, sodium alginate, sodium carboxy methyl cellulose, methyl cellulose, and croscarmellose sodium, crospovidone, and crosslinked forms of polyvinyl pyrrolidone, and any combinations thereof.

Lubricants and glidants may be added to oral solid dosage formulations to enhance powder flow by reducing inter-particle friction. Suitable examples of lubricants include, but are not limited to, magnesium stearate, calcium stearate, stearic acid, sodium stearyl fumarate, talc and any combination thereof.

Suitable examples of glidants include, but are not limited to, metal silicates, silicon dioxides such as colloidal anhydrous silica, higher fatty acid metal salts, metal oxides, alkaline earth metal salts, metal hydroxides, and any combination thereof.

Preservatives may be added to oral solid dosage formulations to prolong storage life of said formulation by reducing degradation and alteration of the active ingredient over time. Suitable examples of preservatives include, but are not limited to, sulphites, benzalkonium chloride, methyl paraben, propyl paraben, benzyl alcohol, sodium benzoate, and any combination thereof.

Antioxidants are a class of preservatives which inhibit the oxidation of other molecules. Suitable examples of antioxidants include, but are not limited to, phenolic-based antioxidants such as butylated hydroxyanisole (BHA), butylated hydroxytoluene (BHT), tert-butyl-hydroquinone (TBHQ), 4-hydroxymethyl-2,6-di-tert-butylphenol (HMBP), 2,4,5-trihydroxy-butyrophenone (THBP), propyl gallate (PG), triamyl gallate, gallic acid (GA), tocopherol acetate, reducing agents such as L-ascorbic acid (vitamin C), L-ascorbyl palmitate, L-ascorbyl stearate, thioglycolic acid (TGA) ascorbyl palmitate (ASP), sulphite-based antioxidants such as sodium sulphite, sodium metabisulphite, sodium bisulphite and thioglycerol and other agents such as disodium ethylenediamine tetraacetate (EDTA), sodium pyrophosphate, sodium metaphosphate, methionine, erythorbic acid and lecithin and any combination thereof.

Surfactants may be used to increase the rate of dissolution of the solid self-emulsifying composition by facilitating wetting thereof. Suitable examples of surfactants include fatty acid and alkyl sulfonates, benzalkonium chloride, dioctyl sodium sulfosuccinate, polyoxyethylene sorbitan fatty acid esters, natural surfactants such as sodium taurocholic acid, 1-palmitoyl-2-oleoyl-sn-glycero-3-phosphocholine, lecithin, and other phospholipids and mono- and diglycerides, and any combination thereof.

Effervescent excipients may be used in powders and tablets in combination with acidic agents to cause a reaction that produces carbon dioxide. Suitable examples of effervescent excipients include sodium bicarbonate, potassium bicarbonate, magnesium bicarbonate, ammonium bicarbonate. The effervescent excipients may be combined with acidic agents, typically weak organic acids such as citric acid and/or ascorbic acid.

The oral solid dosage formulations may additionally include inorganic salts such as sodium chloride, potassium chloride, calcium chloride, sodium phosphate, potassium phosphate, sodium bicarbonate and organic salts such as sodium citrate, potassium citrate, sodium acetate and so forth.

In some embodiments, the oral solid dosage formulation as described herein may comprise an amount of the at least one cannabinoid that is up to about 40 wt %. In some embodiments, the amount of the at least one cannabinoid comprised in the oral solid dosage formulation may be up to at least 1 wt %, 5 wt %, 10 wt %, 15 wt %, 20 wt %, 25 wt %, 30 wt %, 35 wt % and 40 wt % and further in a range of at least 0.1-1 wt %, 1-5 wt %, 5-10 wt %, 10-15 wt %, 15-20 wt %, 20-25 wt %, 25-30 wt %, 30-35 wt %, 35-40 wt %.

In some particular embodiments, the oral solid dosage formulation may comprise CBD in an amount from about 0.5 mg to about 200 mg, or from about 2.5 mg to about 20 mg.

In some particular embodiments, the oral solid dosage formulation may comprise THC in an amount from about 0.5 mg to about 200 mg, or from about 2.5 mg to about 20 mg.

In some embodiments, the oral solid dosage formulation as described herein may comprise the lipophilic solvent in an amount from about 2 wt %, 5 wt %, 10 wt %, 15 wt %, 20 wt %, 25 wt %, 30 wt % and 35 wt %, and further in a range of at least 2-5 wt %. 5-10 wt %, 10-15 wt %, 15-20 wt %, 20-25 wt %, 25-30 wt %, 30-35 wt %.

In some embodiments, the oral solid dosage formulation as described herein may comprise the emulsifier in an amount of from about 1 wt %, 5 wt %, 10 wt %, 15 wt %, 20 wt %, 25 wt % and 30 wt %, and further in a range of at least 0.1-1 wt %, 1-5 wt %, 5-10 wt %, 10-15 wt %, 15-20 wt %, 20-25 wt %, 25-30 wt %.

In some embodiments, the oral solid dosage formulation as described herein may comprise the adsorbent in an amount from about 4 wt %, 8 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, and 80 wt %, and further in a range of at least 4-8 wt %, 8-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt %, 65-70 wt %, 70-75 wt %, 75-80 wt %.

In some embodiments, the oral solid dosage formulation as described herein may comprise the antioxidant in an amount from about 0.01 wt %, 0.05 wt %, 0.1 wt %, 0.2 wt %, 0.3 wt %, 0.4 wt %, 0.5 wt %, 0.6 wt %, 0.7 wt %, 0.8 wt %, 0.9 wt % and 1.0 wt %.

In some embodiments, the oral solid dosage formulation as described herein may comprise the filler and/or binder in an amount from 10 wt %, 15 wt %, 20 wt %, 25 wt %, 30 wt %, 35 wt %, 40 wt %, 45 wt %, 50 wt %, 60 wt % and further in a range of at least 5-10 wt %, 10-15 wt %, 15-20 wt %, 20-25 wt %, 25-30 wt %, 30-35 wt %, 35-40 wt %, 40-45 wt %, 45-50 wt %, 50-60 wt %.

In some embodiments, the oral solid dosage formulation as described herein may comprise the anti-caking agent in an amount from 5 wt %, 10 wt %, 15 wt %, 20 wt %, 25 wt %, 30 wt %, 35 wt %, 40 wt %, 45 wt %, 50 wt % and further in a range of at least 5-10 wt %, 10-15 wt %, 15-20 wt %, 20-25 wt %, 25-30 wt %, 30-35 wt %, 35-40 wt %, 40-45 wt %.

In some embodiments, the oral solid dosage formulation as described herein may comprise the surfactant in an amount from 1 wt %, 5 wt %, 10 wt %, 15 wt %, 20 wt %, 25 wt %, 30 wt %, and further in a range of at least 1-5 wt %, 5-10 wt %, 10-15 wt %, 15-20 wt %, 20-25 wt %, 25-30 wt %.

In some embodiments, the oral solid dosage formulation as described herein may comprise the glidant and/or lubricant in an amount from about 0.1 wt %, 0.2 wt %, 0.5 wt %, 1.0 wt %, 2 wt %, 3 wt %, 4 wt %, 5 wt %.

In some embodiments, the oral solid dosage formulation as described herein may comprise the disintegrant in an amount from about 0.1 wt %, 0.2 wt %, 0.3 wt %, 0.4 wt %, 0.5 wt %, 0.6 wt %, 0.7 wt %, 0.8 wt %, 0.9 wt % and 1.0 wt %.

The compositions as disclosed herein are also advantageous because they can be readily formulated into veterinarily acceptable oral solid dosage formulations suitable for administration to a non-human animal in need of treatment for anxiety or nausea, thereby providing certainty with regard to administration of a pre-defined therapeutically effective amount of the at least one cannabinoid. Suitable non-human animals include, but are not limited to, companion animals such as cats and dogs, laboratory animals such as apes, monkeys, rabbits and rodents, livestock such as cattle, sheep, goats, swine or deer, and zoo animals.

The veterinarily acceptable oral solid dosage formulations comprise the solid self-emulsifying compositions as described herein and physiologically acceptable carriers. The term “physiologically acceptable excipients” as used herein includes one or more of diluents, binders, desiccants, disintegrants, colouring agents, flavouring agents, stabilizers, lubricants/glidants, plasticizers and preservatives, suitable for an oral solid dosage form and non-human mammal animals. The excipients are selected based on the desired physical aspects of the final oral solid dosage formulations and may be present in the same amounts as described above.

Suitable disintegrants may include croscarmellose sodium, one or more of sodium starch glycolate, crospovidone, low substituted hydroxypropyl cellulose, and mixtures thereof.

Suitable binders may include one or more of methyl cellulose, hydroxypropyl cellulose, hydroxypropyl methylcellulose, polyvinylpyrrolidone, gelatin, gum Arabic, ethyl cellulose, polyvinyl alcohol, pullulan, pregelatinized starch, agar, tragacanth, sodium alginate, and mixtures thereof.

Suitable diluents may include one or more of cellulose powdered, dextrates, dextrins, dextrose excipients, fructose, kaolin, lactitol, lactose, mannitol, sorbitol, starch, starch pregelatinized, sucrose, sugar compressible, sugar confectioners, and mixtures thereof.

Suitable lubricants and/or glidants may include colloidal anhydrous silica, one or more of magnesium stearate, stearic acid, magnesium stearate, calcium stearate, talc, hydrogenated castor oil, sucrose esters of fatty acid, microcrystalline wax, yellow beeswax, white beeswax, and mixtures thereof.

The term “flavouring agent(s)” as used herein refers to an ingredient or a compound which may be added to the veterinarily acceptable oral solid dosage formulations to improve palatability to the non-human animal subject and may take the form of proteins, fats, carbohydrates, yeasts or a mixture thereof. Suitable flavouring agents include, but are not limited to, artificial or natural beef flavours, artificial or natural chicken flavours, pork liver extract, artificial meat flavour, honey, yeast, malt, and so forth. The flavouring agents may be present in the veterinarily acceptable oral solid dosage formulations in an amount of 10-40 wt %, based on the total weight of said solid oral dosage formulation, in particular 20-30 wt %, or 20-25 wt %.

### Method for Preparing Solid Self-Emulsifying Composition

The present disclosures provides methods for preparing the solid self-emulsifying compositions and the oral solid dosage formulations as described above.

In some embodiments, the method of preparing a solid self-emulsifying composition comprises:
a) providing a solution of at least one cannabinoid and an emulsifier in a lipophilic solvent;
b) mixing said solution with an adsorbent to produce the solid self-emulsifying composition.

It will be appreciated that the lipophilic solvent comprises a pre-defined amount of the at least one cannabinoid. The pre-defined amount of said cannabinoid may be determined by assaying the lipophilic solution by analytical techniques well known to those in the art such as high pressure liquid chromatography (HPLC), gas chromatography (GC), gas chromatography-mass spectrometry, liquid chromatography-NMR spectroscopy and so forth.

The mixing step may comprise adding said solution dropwise to the adsorbent with continuous stirring and blending the resulting mixture for a period sufficient to obtain the solid self-emulsifying composition, wherein said composition is a free flowing powder or a crystalline powder. Said solution may be added dropwise to the adsorbent with a conventional dripper provided with an orifice size of between 0.3 mm to 2 mm.

The adsorbent may be stirred with an impellor or with a high shear mixer blender. The rate of adding said solution to the adsorbent may be between 60-360 drops per minute. The rate of continuous stirring may be from 50 to 400 rpm. The mixture may be blended at a rate of 100-1000 rpm for 5-60 minutes. The mixture may be held between 30 min and 12 hours before processing in order for droplets of solution to settle within pores of the adsorbent.

Alternatively, the mixing step may comprise spraying the solution onto the adsorbent with continuous stirring and blending the resulting mixture for a period sufficient to obtain the solid self-emulsifying composition, wherein said composition is a free flowing powder or a crystalline powder.

The resulting solid self-emulsifying composition may then be sieved through a classifying sieve, typically from 200 micron to 1000 micron, in particular 425 micron. One or more excipients as described above may be blended with the solid self-emulsifying composition in a conventional blender operated at a chopper speed of from 100 rpm to 500 rpm for a period up to 15 minutes.

In some embodiments, the method further comprises blending the solid self-emulsifying composition with one or more excipients as described above.

### Kit

As an alternative to oral solid dosage formulations, the solid self-emulsifying composition may be conveniently provided in the form of a kit comprising at least one container comprising a pre-defined amount of the solid self-emulsifying composition. The container may have a plurality of compartments configured to contain a plurality of pre-defined amounts of said composition. The pre-defined amounts of said composition may be the same or different, thereby facilitating administration of different dosages or administration of the same dosages at different intervals. Furthermore, at least one of the plurality of compartments of the container may additionally or alternatively contain a nutrient or therapeutic agent. Furthermore, at least one of the plurality of compartments may additionally or alternatively contain a flavoured powder or a flavoured liquid media to improve the flavour of the solid self-emulsifying composition, thereby aiding administration of the solid self-emulsifying composition.

The kit may be used to achieve a controlled therapeutic effect whereby the dosage may be tailored to the subject's needs or symptoms and thereby administer a therapeutically effective amount of the at least one cannabinoid, optionally in combination with a nutrient or therapeutic agent.

The nutrient may be a vitamin, vitamin supplements such as omega fatty acids, omega-3-fatty acids, or a mineral.

The therapeutic agent may be a drug or a combination of drugs selected for their clinical effects and applicability to treatment of a human disease, condition or disorder. Such drugs may be selected from analgesics, antacids, antianxiety drugs, anti-arrhythmics, anti-bacterials, anti-biotics, anti-micotics, anti-coagulants and thrombolytics, anti-convulsants, anti-depressants, anti-diarrheals, anti-emetics, anti-epileptics, anti-fungals, anti-histamines, anti-hypertensives, anti-inflammatoirenti-neoplastics, anti-psychotics, anti-pyretics, anti-spastics, anti-virals, barbiturates, beta-blockers, bronchodilators, cold cures, cholesterol lowering drugs, corticosteroids, cough suppressants, cytotoxics, decongestants, diuretics, expectorant, hormones, hypoglycemics, immune-suppressives, laxatives, muscle relaxants, sedatives, sex hormones, sleeping drugs, oncolytics, and tranquilizers.

### Clinical Applications

It will be appreciated that the solid self-emulsifying composition or the oral solid dosage formulations as described herein may be used therapeutically, in other words to prevent, treat and/or lessen the severity of a wide range of human diseases, conditions and disorders, including inflammatory, neurological, psychiatric disorders, malignancies and further immune, metabolic disorders, nutritional deficiencies, infectious diseases, and types of gastrointestinal disorders, cardiovascular disorders, and various types of pain, including chronic and neuropathic pain.

In view of the current understanding regarding clinical applications of cannabinoids in patients, the solid self-emulsifying compositions or oral solid dosage formulations as described herein may be applicable, although not only, to at least one of depression, sleeping disorders, eating disorders, cancer, multiple sclerosis, graft versus host disease (GVHD), Parkinson's, epilepsy, autism, tuberculosis, ulcerative colitis, morbus Crohn, inflammatory bowel disorder (IBD), irritable bowel syndrome (IBS), appetite stimulant, appetite depressant, obesity, nausea, neuropathic pain, anxiety, Alzheimer's disease, amyotrophic lateral sclerosis (ALS), gastrointestinal disorders, hypertension, incontinence, pruritus, arthritis, arthrosis, rheumatic inflammation, insomnia, mycosis, local and/or chronic pain, inflammation, attention deficit and hyperactivity disorder (ADDH), vomiting, atopic dermatitis, fibromyalgia, autoimmune deficiency syndrome (AIDS), mood disorders, erectile dysfunction, cancer, premature ejaculation, bone growth, treatment resistant epilepsy, in particular treatment resistant childhood epilepsy.

The dose or frequency of administering the solid self-emulsifying composition or the oral dosage formulation as described herein would be readily determined by those skilled in the art and is dependent on the age, weight, general physical condition, or other clinical symptoms specific to the subject to be treated.

The bioavailability of the at least one cannabinoid in the solid self-emulsifying composition or the oral dosage formulation as described herein is improved in comparison to currently available oral dosage formulations of cannabinoids, thereby resulting in an improved pharmacokinetic profile of the at least one cannabinoid. The term “improved pharmacokinetic profile” as used herein refers to one or more of the following criteria in comparison to conventional oral dosage formulations of one or more cannabinoids: 1) high average Cₘₐₓ; 2) pharmacokinetic parameters with reduced variation; and/or 3) reduced effective dose.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

The following examples are to be understood as illustrative only. They should therefore not be construed as limiting the invention in any way.

Oral Solid Dosage Formulations including the amounts of active ingredients and excipients as described in the tables below were prepared according to the following procedures.

A required amount of cannabinoid resin containing CBD, THC or a mixture of CBD:THC) is placed into a stainless steel or glass container. Approximately half of the required amount of medium chain triglyceride (MCT) is transferred into the container with the resin and a suitable size magnetic stirring bar or blender is placed into the container. The mixture is stirred slowly at a speed which avoids formation of a vortex until the resin has dissolved. Vitamin E is then added into the container, followed by the remainder of the MCT, and the mixture is blended until homogenous.

The required amount of a polyoxyl castor oil (e.g. Kolliphor EL″) is added to the lipophilic solution of the cannabinoid(s) and blended for 10-30 minutes at a speed which avoids vortexing.

The required amount of colloidal anhydrous silica is transferred to the blender and the pre-defined amount of lipophilic solution of the cannabinoid(s) is added dropwise at a rate of approximately 60-600 drops per minute to the silica which is under continuous stirring at an impeller speed of between 50 to 400 rpm.

After the lipophilic solution has been added, the mixture is blended for about 10 minutes at an impeller speed of 100-1000 rpm. Any lumps may be removed by passing the solid self-emulsifying composition through a 425 micron aperture sieve.

Prior to addition of the excipients, each of the dry powder excipients is sieved through a 425 micron aperture sieve. The pre-sieved microcrystalline cellulose and tricalcium phosphate are transferred to the blender and spread over the solid self-emulsifying composition bed.

The impeller speed is then set to between 200 and 500 rpm and the mixture is blended for approximately 15-30 minutes until a homogeneous blend is achieved. The pre-sieved magnesium stearate is then transferred to the blender, the impeller speed is set to between 200 and 500 rpm and the mixture is blended for 120 to 300 seconds.

The final master blend is then discharged into a suitable intermediate bulk container for transport and/or storage.

The final master blend may be processed in an encapsulation machine to fill hard shell hydroxypropyl methylcellulose capsules. The filled hard shell hydroxypropyl methylcellulose capsules may then be packaged in blister packs and further packaged in approved size “shelf-ready” cartons.

Oral solid Dosage Formulation 1

| | QTY per | |
|---|---|---|
| Ingredients | Capsule, mg | % |
| Colloidal Anhydrous Silica | 60 | 22.22 |
| (Aeroperl 300 Pharma) | | |
| Medium Chain Triglycerides | 57.5 | 21.30 |
| API - CBD or THC | 2.5 | 0.93 |
| Polyoxyl castor oil | 12 | 4.44 |
| Cellulose microcrystalline | 94 | 34.81 |
| Tricalcium Phosphate Granular | 42 | 15.56 |
| Sodium stearyl fumarate | 2 | 0.74 |
| TOTAL | 270.00 | 100.00 |

Oral solid Dosage Formulation 2

| | QTY per | |
|---|---|---|
| Ingredient | Capsule, mg | % |
| Colloidal Anhydrous Silica | 60 | 22.22 |
| (Aeroperl 300 Pharma) | | |
| Medium Chain Triglycerides | 55 | 20.37 |
| API - 2.5 mg CBD:2.5 mg THC | 5 | 1.85 |
| Polyoxyl castor oil | 12 | 4.44 |
| Cellulose microcrystalline | 94 | 34.81 |
| Tricalcium Phosphate Granular | 42 | 15.56 |
| Sodium stearyl fumarate | 2 | 0.74 |
| TOTAL | 270.00 | 100.00 |

Oral solid Dosage Formulation 3

| | QTY per | |
|---|---|---|
| Ingredient | Capsule, mg | % |
| Colloidal Anhydrous Silica | 60 | 22.22 |
| (Aeroperl 300 Pharma) | | |
| Medium Chain Triglycerides | 50 | 18.52 |
| API - CBD or THC | 10 | 3.70 |
| Polyoxyl castor oil | 12 | 4.44 |
| Cellulose microcrystalline | 94 | 34.81 |
| Tricalcium Phosphate Granular | 42 | 15.56 |
| Sodium stearyl fumarate | 2 | 0.74 |
| TOTAL | 270.00 | 100.00 |

Solid Oral Dosage Formulation 4

| | QTY per | |
|---|---|---|
| Ingredient | Capsule, mg | % |
| Colloidal Anhydrous Silica | 60 | 22.22 |
| (Aeroperl 300 Pharma) | | |
| Medium Chain Triglycerides | 40 | 14.81 |
| API - 10 mg CBD:10 mg THC | 20 | 7.41 |
| Polyoxyl castor oil | 12 | 4.44 |
| Cellulose microcrystalline | 94 | 34.81 |
| Tricalcium Phosphate Granular | 42 | 15.56 |
| Sodium stearyl fumarate | 2 | 0.74 |
| TOTAL | 270.00 | 100.00 |

Oral solid Dosage Formulation 5

| | QTY per | |
|---|---|---|
| Ingredient | Capsule, mg | % |
| Colloidal Anhydrous Silica | 60 | 22.22 |
| (Aeroperl 300 Pharma) | | |
| Medium Chain Triglycerides | 45 | 16.67 |
| API - 5 mg CBD:10 mg THC | 15 | 5.55 |
| Polyoxyl castor oil | 12 | 4.44 |
| Cellulose microcrystalline | 94 | 34.81 |
| Tricalcium Phosphate Granular | 42 | 15.56 |
| Sodium stearyl fumarate | 2 | 0.74 |
| TOTAL | 270.00 | 100.00 |

1-47. (canceled)

48. A solid self-emulsifying composition for oral solid dosages comprising at least one cannabinoid, a lipophilic solvent, an emulsifier, and an adsorbent, wherein the adsorbent is a mesoporous inert particulate material.

49. The composition according to claim 48, wherein the at least one cannabinoid may be selected from a group comprising anandamide, 2-arachidonoylglycerol, cannabichromene (CBC), cannabichromenic acid (CBCA), cannabichormevarin (CBCV), cannabichromevarinic acid (CBCVA), cannabidiol (CBD), cannabidiolic acid (CBDA), cannabidivarin (CBDV), cannabidivarinic acid (CBDVA), cannabielsoin (CBE), cannabicyclol (CBL), cannabinodiol (CBND), cannabigerol (CBG), cannabigerolic acid (CBGA), cannabigerovarin (CBGV), cannabigerovarinic acid (CBGVA), cannabinol (CBN), cannabinolic acid (CBNA), cannabitriol (CBT), delta-8-tetrahydrocanninol, delta-8-tetrahydrocannabinolic acid, delta-9-tetrahydrocannabinol (THC), delta-9-tetrahydrocannabinolic acid (THCA), delta-9-tetrahydrocannabivarin (THCV), delta-9-tetrahydrocannabivarinic acid (THCVA), 11-nor-9-carboxy-delta-9-tetrahydrocannabinol (THCCOOCH), 11-nor-9-carboxy-delta-8-tetrahydrocannabinol, 11-hydroxy-delta-8-tetrahydro-cannabinol, and 11-hydroxy-delta-9-tetrahydrocannabinol, dimethyl heptylpentyl cannabidiol (DMHP-CBD), 6,12-dihydro-6-hydroxy-cannabidiol, (3S,4R)-7-hydroxy-.DELTA.6-tetrahydrocannabinol homologs and derivatives, (+)-4-[4-DMH-2,6-diacetoxy-phenyl]-2-carboxy-6,6-dimethylbicyclo[3.1.1]he-pt-2-en, and other 4-phenylpinene derivatives, and cannabidiol (−)(CBD) analogs such as (−)CBD-monomethylether, (−)CBD dimethyl ether; (−)CBD diacetate; (−)3′-acetyl-CBD monoacetate, cannabinol propyl variant (CBNV), and nabilone.

50. The composition according to claim 49, wherein the cannabinoid comprises cannabidiol (CBD).

51. The composition according to claim 49, wherein the cannabinoid comprises delta-9-tetrahydrocannabinol (THC).

52. The composition according to claim 49, wherein the cannabinoid comprises CBD and THC in a ratio of from 1:100 to 100:1.

53. The composition according to claim 48, wherein the lipophilic solvent comprises a vegetable oil, medium chain triglycerides, or a mixture thereof.

54. The composition according to claim 48, wherein the emulsifier comprises a surfactant.

55. The composition according to claim 54, wherein the emulsifier comprises a non-ionic surfactant.

56. The composition according to claim 54, wherein the emulsifier comprises a polyethoxylated non-ionic surfactant.

57. The composition according to claim 48, wherein the mesoporous inert particulate material has a particle size in a range of from about 3 micron to about 350 micron.

58. The composition according to claim 57, wherein the mesoporous inert particulate material has a particle size in a range of from about 20 micron to about 60 micron.

59. The composition according to claim 48, wherein the inert particulate material is mesoporous with a mesopore volume in a range of from about 1.5 to about 1.9 mL/g.

60. The composition according to claim 48, wherein the mesoporous inert particulate material has a surface area of from about 150 to 350 m²/g.

61. The composition according to claim 48, wherein the mesoporous inert particulate material comprises amorphous silica.

62. The composition according to claim 48, further comprising a lipophilic antioxidant.

63. A method of preventing, treating and/or lessening the severity of a disease in a subject, wherein the disease is at least one of an inflammatory disorder, a neurological disorder, a psychiatric disorder, a malignancy, an immune disorder, a metabolic disorder, a nutritional deficiency, an infectious disease, a gastrointestinal disorder, a cardiovascular disorder, cancer, pain said method comprising administering to a subject in need thereof an effective amount of the solid self-emulsifying composition according to claim 48.

64. A method of providing a plasma concentration of at least one cannabinoid in a pre-determined concentration range in a subject comprising administering to the subject a pre-determined amount of the solid self-emulsifying composition according to claim 48.

65. The composition according to claim 61, wherein the amorphous silica is present in an amount from about 4 wt % to about 80 wt % of the composition.

66. The composition according to claim 65, wherein the emulsifier is present in an amount from about 2 wt % to about 35 wt % of the composition.

67. The composition according to claim 66, wherein the lipophilic solvent is present in an amount greater than about 5 wt % of the composition.
